SEQUENCE LISTING (16 sequences)

SEQ ID NO: 1 - DNA, 243 bp, Artificial Sequence
  936 amplicon consensus sequence
aaggtctattgtggtattccgattttagtaacagcttatgatagtaaacttatcttattccgttcaatagctattaaattgctagaaaaaaatggtattaaagctgacgaaacaagtgtattagtgaaagactttatctcttgttattgtcggcttaatattgttgatgaaccagcagaacaatggcgaaatgctgaaatgaaacgtttggcttctttgcaagagttaatgtattatggaggc

SEQ ID NO: 2 - DNA, 196 bp, Artificial Sequence
  C2 amplicon consensus sequence
gctcgtgaaattgacggagtaggctattataaagatgttactattacggaggtatcactaactccgttaccaagtaataaaggtgctaaagtgacaaaagtaagagaagaagaaaaaggagaacaaaaacaaatgggtgcaaacgaaacacaagaaatcatgaagcaagcaattgaagcaggtgtaaaagttcgag

SEQ ID NO: 3 - DNA, 123 bp, Artificial Sequence
  P335 amplicon consensus sequence
tgaaacagttacgattcaacctgatgaaattaaaaaggtaagcactgggctagctgttcaacttggtgatgatgaagtattgaaattatacgaccgttcaagtaatccagttaagcgtggcat

SEQ ID NO: 4 - DNA, 171 bp, Artificial Sequence
  Cos amplicon consensus sequence
ggttcacgtgtttatgaaaaatggactgatattacaggtcttgctaatattgatgatgaagcaggtaaaattgctgatattgatgatccaaaactttctcttatcaaatacactatcaagcgctatgctggtatctcaacagttactaacagcttgcttgctgattctgct

SEQ ID NO: 5 - DNA, 278 bp, Artificial Sequence
  Pac amplicon consensus sequence
aatggaaacagataacacaacagtcgaaacggtcgaaactgaggaagtaagccatgacgttgatgaaaatcaatcgagcgacttccaagcgccgcaatcacagtcagaacgagatagcattgttaacaaggcagtccaaaaagccttgagtaattataagaagggcgaagaaactcgaatcaatgaggcaatcgctaaagcgttaaagaaagaacaagactattctaaattatcggctgctgagcgggctagcaaggaatttgaagaccaaaaagcag

SEQ ID NO: 6 - DNA, 24 bp, Artificial Sequence
  Forward primer 936
aaggtctattgtggtattccgatt

SEQ ID NO: 7 - DNA, 25 bp, Artificial Sequence
  Reverse primer 936
acctccataatacattaactcttgc

SEQ ID NO: 8 - DNA, 21 bp, Artificial Sequence
  Forward primer c2
gctcgtgaaattgacggagta

SEQ ID NO: 9 - DNA, 22 bp, Artificial Sequence
  Reverse primer c2
ctcgaacttttacacctgcttc

SEQ ID NO: 10 - DNA, 23 bp, Artificial Sequence
  Forward primer p335
tgaaacagttacgattcaacctg

SEQ ID NO: 11 - DNA, 20 bp, Artificial Sequence
  Reverse primer p335
atgccacgcttaactggatt

SEQ ID NO: 12 - DNA, 24 bp, Artificial Sequence
  Forward primer Cos
ggttcacgtgtttatgaaaaatgg

SEQ ID NO: 13 - DNA, 24 bp, Artificial Sequence
  Reverse primer Cos
agcagaatcagcaagcaagctgtt

SEQ ID NO: 14 - DNA, 24 bp, Artificial Sequence
  Forward primer Pac
aatggaaacagataacacaacagt

SEQ ID NO: 15 - DNA, 23 bp, Artificial Sequence
  Reverse primer Pac
tctgctttttggtcttcaaattc

SEQ ID NO: 16 - protein, 87 aa, Artificial Sequence
  structural protein 1 (GenBank ASZ71906.1)
MIDYIKVYCGIPILVTAYDSKLILFRSIAIKLLEKNGIKADETSVLVKDFISCYCRLNIVDEPAEQWRNAEMKRLASLQELMYYGGI

(19) United States
(12) Patent Application Publication
(10) Pub. No.: US 2022/0205056 A1
(43) Pub. Date: Jun. 30, 2022

(54) PHAGE TEST KIT

(71) Applicant: DSM IP Assets B.V., Heerlen (NL)

(72) Inventors: Paulus Petrus DE WAAL, Echt (NL); Laurens Leendert HANEMAAIJER, Echt (NL)

(21) Appl. No.: 17/603,789

(22) PCT Filed: Apr. 15, 2020
(86) PCT No.: PCT/EP2020/060513

§ 371 Date: Oct. 14, 2021

(30) Foreign Application Priority Data
Apr. 18, 2019 (EP) 19170078.0

(51) Int. Cl. C12Q 1/70; C12Q 1/686; A23C 7/00; G01N 33/04

(52) U.S. Cl. CPC C12Q 1/701; C12Q 1/686; C12Q 2600/16; G01N 33/04; A23C 7/00

(57) ABSTRACT

The present invention relates to a kit suitable for detection and quantification of phage DNA from a lactic acid bacteria infecting phage in a dairy sample. The invention provides a quantitative polymerase chain reaction (qPCR) kit for detection and quantification of phage DNA to from a lactic acid bacteria infecting phage in a dairy sample, said kit comprising a first primer pair and wherein said first primer pair has a robustness of a delta Cq lower than 1.0 cycle when tested in a temperature range of 55.0-70.0, preferably 55.0-68.0, more preferably 58.6-65.6 degrees Celsius and wherein said first primer pair is directed to a lactic acid bacteria infecting phage which is a lactococcal phage from the subgroup 9362, c2 or P335 or a streptococcal phage from the subgroup pac.

### FIELD

The present invention relates to a kit suitable for detection and quantification of phage DNA from a lactic acid bacteria-infecting phage in a dairy sample.

### BACKGROUND AND INTRODUCTION INTO THE INVENTION

The presence of bacteriophages, henceforth called phages, in industrial dairy environment fermentations is generally considered to negatively impact production, with phage infection affecting the rheological and textural properties of the end product. It has been postulated that the main source of new phages entering a product line is raw milk. The first step in the amelioration of this problem is usually the detection and/or quantification of the phages in question. Currently, phage detection is essential to confirm that fermentation slow-down or failure has indeed been caused by the presence of phages but due to the traditionally used methods remains retrospective.

At present phage detection and quantification is done by using a classical overlay-method or performing an acidification-assay, the former supplying information on phage levels expressed as plaque-forming units (PFU) per millilitre, the latter supplying information on the effect of the phage titres present in a dairy sample on acidification of the strains, which is the primary performance criterium in a dairy fermentation process. In this overlay technique the phage is allowed to propagate in a confluent lawn of bacterial host cells immobilized in a thin and soft layer of top agar, in which a circular transparent area of lysed cells (i.e. the ‘plaque’) will develop, resulting from a series of phage-infection, phage-multiplication, phage-liberation chain reaction events. However, the effectiveness of the plaque assay to monitor phage is dependent not only upon the phage and the bacterial strain; plaque formation is highly influenced by the (biological) physical and chemical conditions. Accordingly, when propagating phage in a suboptimal environment, plaques may fail to appear. In the acidification-assay a phage is allowed to propagate in liquid medium with a bacterial host and compared with a control only containing liquid medium with the bacterial host. From both, pH is monitored with a pH-indicator or pH-probe. If pH is affected, i.e. pH remains higher compared to control this indicates enough virulent phages were present to affect the culture. Also, this method has its disadvantages and the effectiveness for measuring a pH effect caused by phages depends on (biological) physical and chemical conditions in the liquid medium. When performing an acidification assay under sub-optimal conditions phages can be present without affecting pH, leading to a false negative result. Both methods are time-consuming and labour-intensive. Therefore, only retrospectively such methods establish a phage was causing the fermentation failure rather than enable preventive actions for a dairy producer to suppress the phage titers from causing these failures. Fermentation failures come with a serious economic loss for the dairy producer because of down-grading to less valuable products.

Therefore, a clear need exists for a fast test to detect, classify and quantify phages in a dairy process. This enables the dairy producer to take immediate corrective actions such as heat treatment, apply proper sanitation procedures, or introduce another phage-unrelated rotation culture to suppress the risen phage levels. Currently, for a wide variety of food applications, real-time quantitative polymerase chain reaction has emerged as a method of choice to identify and quantify microbe (contaminant) species due to its rapid and sensitive identification capabilities. Furthermore, recent advances in DNA Polymerase technology (such as polymerase Sso7d-fusion polymerase SsoAdvanced™, Biorad) and optimization of reaction conditions (e.g. intensity and stability of fluorophores such as SYBR-related dyes), opened up possibilities for a fast real-time o quantitative polymerase chain reaction protocol (results within one hour) and direct real-time quantitative polymerase chain reaction on matrices containing known PCR-inhibitors without elaborate DNA extraction protocols or samples processing. For setting up a real-time quantitative polymerase chain reaction for detection of phage DNA one has to consider the different most prevalent phage subgroups in the dairy environment. The lytic 936, c2 phages and the lysogenic P335 phages cause the most pressing concern for lactococci cells in a starter culture. For thermophile streptococci in the starter cultures, virulent phages are subdivided based on mode of DNA packaging in the isometric head of the phage, namely the cos and pac Streptococcus thermophilus phages. In recent years two more streptococcal phage groups were identified, i.e. 5093 and 987 phages. In summary, the criteria for a dairy producer to use a phage test kit for fast decision making or point-of-care test would be the following:

(A) detection of at least one, preferably at least 2, 3 or 4 and most preferably all phages within the prevalent subgroups which would call for conserved oligonucleotide primers, and optionally a probe, for each subgroup of phages. Suitable primers and optionally probes need to be directed to conserved genomic regions in phage DNA. The term conserved as used in this context refers to genomic regions in different phages having preferably at least 90% nucleotide identity, more preferably a least 95% identity and most preferably at least 98% identity.

(B) a sensitive test with at least one and preferably at least two of the next features: a quantification efficiency, preferably between 90-110%, linear standard curve (R2>0.980), high precision between experimental experiments, consistency across replicate experiments, no primer dimers and a wide dynamic range detecting bacteriophages at least at the same level and preferably below the detection limit of the overlay assay (LOD) 5≤10³ PFU/ml). The quantification efficiency can be determined by applying the developed qPCR assay on a dilution series of the target DNA with at least three concentrations thereof, preferably diluted ten-fold. The determined Cq values for the dilution series are plotted against the concentration or dilution factor of the target DNA on a logarithmic scale. Through these data points a linear regression curve is generated and the slope of the trend line is calculated. The qPCR efficiency can be calculated using the equation: Efficiency=(10^{(-1/Slope})*100. Preferably, qPCR efficiencies range from 90% to 110%.

(C) fast protocol without extensive pre-treatment or DNA purification steps, e.g. only dilution of milk) allowing for results within one or two hours.

Since dairy matrices are known to contain inhibiting compounds for efficient PCR, especially for reliable quantification, the use of a robust PCR-polymerase-primer reaction mixture seems to be a prerequisite for the success of such a kit in the dairy environment.

PCR detection of dairy phage subgroups in (processed) dairy samples has been reported in the prior art, even in a multiplex manner (i.e. multiple primer sets targeting a range of distinct phage species in one dairy sample). Labrie and Moinaeu (2000, Appl Environ Microbiol. Vol. 66: pp. 987-994.) set up a multiplex PCR assay to detect c2, 936 and P335 subgroups of lactococcal phages in one PCR reaction using whey (powders) as dairy sample. The detection was based on primer design yielding different sized amplicons for each subgroup making interpretation of the presence of each phage group in the sample possible with standard gel electrophoresis. However, the assay purely gives a qualitative result of the presence of a certain phage group or phage groups in the dairy process, but no quantification of phage titers which is needed to indicate the severity of the actual phage problem. Binetti et al. (2005; Detection and characterization of Streptococcus thermophilus bacteriophages by use of the antireceptor gene sequence. Appl Environ Microbiol. 71: 6096-6103) developed a PCR detection method for Streptococcus thermophilus phages based on targeting VR2, a variable region of the antireceptor gene claimed by authors to be conserved in all S. thermophilus phages. The assay worked directly on milk samples without pre-treatment or need for DNA purification steps spiked with S. thermophilus phages and showed a detection limit of 10⁵ PFU/ml. Again, the assay developed only gave a qualitative result here. Similarly, as Labrie and Moineau, Quiberoni et al. (2006. Diversity of Streptococcus thermophilus Phages in a Large-Production Cheese Factory in Argentina. J. Dairy Sci. 89: 3791-3799) developed a multiplex PCR assay for the detection of S. thermophilus cos and pac phages with one PCR reaction. However, this assay was conducted only on phage lysates for typing isolated phages from infected samples and no quantitative results were given. The phage titers determined by classical overlay assay across whey samples ranged from almost 7.5×10² to 3×10⁵ PFU/mL. Inventors of application WO2006/136640 developed a multi-PCR assay to detect phages virulent against Lactobacillus, Lactococcus (c2, 936, P335) and Streptococcus (cos) based on fragment size, but here also no quantification of phage titers was determined. A similar observation can be made for Ali et al. (2014, African J Microbiology research, Detection and characterization of bacteriophages attacking dairy Streptococcus thermophilus starter cultures, 8: 2598-2603) in which conserved primers for streptococcal cos and pac phages were developed for a multiplex PCR on phages isolated from yoghurt samples. Prior art on the development of real-time quantitative polymerase chain reaction protocols for the quantification of phage titers includes Del Rio et al. (2008, Appl Environ Microbiol., Multiplex fast real-time PCR for quantitative detection and identification of cos- and pac-type Streptococcus thermophilus bacteriophages, 74: 4779-4781) who developed a TaqMan-based (i.e. use of labelled probes) qPCR assay to detect and quantify cos and pac phages directly in artificially spiked ten fold-diluted skimmed milk samples. The LOD of the developed assays by Del Rio et al. (2008) seemed higher than for the standard plaque assay which is reportedly 10³ PFU/mL observing the supplied data. One microliter of an artificially ten-fold diluted spiked milk sample ranging from 10³ to 10⁹ PFU/mL of cos or pac phage was used a template in the qPCR reaction (total volume 20 microliters). The lowest amount detected for the pac assay seemed to be 10² PFU/reaction (or per microliter of sample) with a Cq value of around 30. This translates to a limit of detection of 10⁵ PFU/mL for the pac assay. For the cos assay the lowest amount detected was 10 PFU/reaction with a Cq value of around 30. This translates to a limit of detection of 10⁴ PFU/mL for the cos assay.

Verreault et al. (2011 Detection of airborne lactococcal bacteriophages in cheese manufacturing plants. Appl Environ Microbiol. 77: 491-497) displayed results of a real-time quantitative polymerase chain reaction protocol to quantify lactococcal 936 and C2 phages in surface or air swab samples from a dairy plant. In this assay, LOD and specificity of the assays was tested and found satisfactory. However, samples analyzed (swabs collected in water with Tween) are relevant for phage management in dairy industry, they are not typical such as milk or whey. Those typical matrices are the most challenging matrices because of their inhibiting substances for the real-time quantitative polymerase chain reaction. Therefore, the robustness of the assays by Verreault et al. (2011) on milk matrices remained elusive. Furthermore, no results on PCR efficiency or limit of quantification were presented in that study.

Ly-Chatain et al. (2011, Int J Microbiol., Direct quantitative detection and identification of Lactococcal bacteriophages from milk and whey by real-time PCR: application for the detection of lactococcal bacteriophages in goat's raw milk whey in France, 2011: 594369) presented a qPCR protocol for detecting c2, 936, and P335 lactococcal phages in whey and raw milk samples. Although the developed protocols showed a sufficient LOD related to the overlay assay (10² PFU/mL) and reasonable qPCR efficiencies (94-98%), the protocol included an extraction protocol to isolate phage DNA from whey and milk samples, thereby removing PCR-inhibiting compounds from the dairy matrix. The extraction protocol included steps, such as using a microcentrifuge and spinning at high gravity force (5000 g), using isopropanol and ethanol to precipitate the phage DNA and using reagents from a DNA isolation kit, which are complicating the proposed protocol to be executed at a dairy customer, which do not have these types of equipment or expertise.

Furthermore, the need for specialized molecular biology grade reagents come with added cost to a commercial kit making it less attractive for the dairy customer.

Similarly, Muhammed et al. (2017, PLoS One., A high-throughput qPCR system for simultaneous quantitative detection of dairy Lactococcus lactis and Leuconostoc bacteriophages, 12: e0174223) developed a phage detection/quantification protocol based on multiplex rt-qPCR for c2, 936 and P335 lactococcal phages and additionally Leuconostoc phages with sufficient LOD and qPCR efficiencies for relevant dairy samples. However, similarly as for Ly-Chatain et al. (2011, Int J Microbiol. 2011: 594369) for sample pre-treatment an elaborate phage DNA purification protocol with multiple steps including specialized molecular DNA extraction or pre-amplification kits is necessary prior to conducting the actual qPCR and obtaining the results. Especially, an overnight Dnase-I treatment prohibits the use of this protocol as a fast test or point-of-care test for fast decision making at a dairy plant. The perspective of Muhammed et al. (2017) was actually more to develop such a protocol for high throughput purposes and retrospective analysis of phage dynamics within process streams in a dairy plant.

### FIGURES

FIG. 1: This figure shows the qPCR and overlay results of tenfold serial dilutions of four phage lysates. The order of the figures corresponds with the phage names which are indicated in the o middle. Graphs in the upper part shows the phage particles as determined by qPCR, either for 936 or c2 phages (y-axis; LOG qPCR [particles/mL]) over dilution (x-axis; LOG dilution). Pictures in the lower part shows the plaque forming units (PFU) as determined with the overlay assay over dilution. White circles indicate the highest dilution were single plaques were detected. PFU/ml can be calculated as follows: (−detected dilution)×100, i.e. in the first overlay from the left the highest detected dilution is 10⁻⁷, the number of PFU/ml=−10⁻⁷=10⁷×100=10⁹ PFU/ml

FIG. 2: Each graph represents the phage level development over time for each acidification experiment which was infected with phage. Set-up of the acidification experiment (# indicated above graph), and the identity and titer of the phages infected at TO are listed in Table 10. Upper panel of graphs shows the phage particles as determined by qPCR (y-axis; LOG qPCR [particles/mL]) over time (x-axis; hours). Above each graph in the upper panel is also indicated which qPCR assay was used. Lower panel of graphs shows the phage titers as determined with overlay assay (y-axis; LOG overlay [PFU/mL]) over time (x-axis; hours). Each datapoint represents average value of five measurements. Error bars represent the standard deviation.

### DESCRIPTION OF SEQ ID NUMBERS

SEQ ID NO 1 amplicon 936 phage

SEQ ID NO: 2 amplicon c2 phage

SEQ ID NO: 3 amplicon P335 phage

SEQ ID NO 4 amplicon cos phage

SEQ ID NO: 5 amplicon pac phage

SEQ ID NO: 6 primer 936 phage

SEQ ID NO 7 primer 936 phage

SEQ ID NO: 8 primer c2 phage

SEQ ID NO: 9 primer c2 phage

SEQ ID NO 10 primer P335 phage

SEQ ID NO: 11 primer P335 phage

SEQ ID NO: 12 primer cos phage

SEQ ID NO 13 primer cos phage

SEQ ID NO: 14 primer pac phage

SEQ ID NO: 15 primer pac phage

SEQ ID NO: 16 structural protein 1 (GenBank: ASZ71906.1)

### SUMMARY

The invention of the present application is a phage DNA detection kit with attractive features for use as a fast, user-friendly test at a dairy producer. The dairy samples which can be used with such a kit are for example milk, whey (powder), rinse water, starter media and broth of grown bulk starters. Essentially, a minimal amount of sample treatment is needed for obtaining reliable results o on phage titers, i.e. at most a single dilution step with water. To achieve the above, the fast phage test kit comprises at least one primer pair which, firstly, is able to detect a phage within one of the relevant subgroups of dairy phages (e.g. c2, 936, P335, cos, pac, 5093 and 987) because of the conserved nature of the sequences to which the primers hybridize, and secondly have been selected into the kit because of their robustness, ensuring hybridizing to the target sequence and efficient amplification in the challenging matrix of milk or other dairy process streams such as whey or rinse water resulting in a reliable quantification of the present phage titers.

To achieve such a relevant phage detection and quantification kit, the inventors of the present invention have designed conserved primers based on a phage genome sequences of relevant dairy bacteriophages (of 936, c2, P335, cos, pac, 5093, 987 subgroups). In the case of developing a multiplex qPCR assay targeting multiple sequences in a single assay, the inventors have additionally designed a probe on a conserved sequence between both the forward and reverse primer of the targeted amplicon sequence. Phage genome sequences are found in public databases such as Genbank Nucleotide (https://www.ncbi.nlm.nih.gov/nuccore) or can be generated by collecting bacteriophages from relevant dairy process streams (e.g. whey) and employing next generation sequencing technologies (e.g. Illumina) to extract their genome sequences.

To achieve the design of suitable conserved primers to allow for amplification of specific desired DNA fragments for each subspecies of phages, a set of genome sequences of phages within a subgroup are compared to identify conserved genomic regions within a subspecies of phages. Nucleotide sequences are said to be conserved when exhibiting a certain level of similarity or homology. Two sequences being homologous indicate a common evolutionary origin. Whether homologous sequences are closely related or more distantly related is indicated by “percent identity” or “percent similarity”, which is high or low, respectively. Although disputed, to indicate “percent identity” or “percent similarity”, “level of homology” or “percent homology” are frequently used interchangeably. A comparison of sequences and determination of percent identity between multiple sequences can be accomplished using a mathematical algorithm. The skilled person will be aware of the fact that several different computer programs are available to align multiple sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). For nucleotide sequences, Geneious ClustalW and MAFFT is typically used. For the invention described below such primers are described for 936, c2, P335 and pac phages, for which 17, 13, 17 and 14, respectively, genome sequences of each subspecies were compared to identify such conserved genomic regions. The identified regions are conserved within this set of genomes and are found to be homologous to 95% nucleotide identity. The identified genomic regions for 936, c2, P335 and pac are further described in Example 1 of the invention. Subsequently, oligonucleotide primers are designed within this genomic region. The person skilled in the art is aware of computer programs for primer design (such as for example Primer-BLAST).

Second, the inventors have designed multiple primer sets for each subgroup to test the specificity, efficiency and robustness of such oligonucleotide primer (-and probe) sets. The different sets oligonucleotides primers and optionally labelled probe were combined with a suitable qPCR reaction mixture comprising a DNA polymerase such as for example SsoAdvanced (Biorad), PlatinumTaq (Thermofisher), PowerUp (Thermofisher), dNTPs, MgCl₂, a suitable reaction buffer (e.g. Tris-HCl pH=8.8), water, DNA binding dye or probe. Many commercial ready-made 2×, 5× or 10× concentrated polymerase reaction mixes are available with in the case of use of a DNA binding dye the fluorophore (e.g. SYBR) included, such as LyoGreen™ (Promega), SsoAdvanced™ Universal SYBR® Green Supermix, PowerUp SYBR Green Master Mix (ThermoFisher), Platinum SYBR Green qPCR SuperMix (ThermoFisher). The inventors have tested the specificity and efficiency of the primer (probe) set by applying qPCR on a dilution series of isolated/purified DNA from bacteriophages of the different subgroups and a no-template-control (NTC; using e.g. water as sample) and applying qPCR with the primers in the reaction mixture in a suitable qPCR device (such as Biorad CFXTM systems). Suitable specific primers designed for a specific bacteriophage subgroup show a PCR efficiency of between 90-110% on the dilution series, and for the NTC no signal below 40 cycles in the qPCR. Optionally, different primer concentrations for each primer were tested to improve the robustness of the assay. Furthermore, an important analysis for a commercial phage test kit is that the different primer sets are tested for robustness by subjecting the same relevant phage DNA sample for qPCR amplification with a range of annealing temperatures in the programmed PCR cycle conditions. For instance, a temperature range of 55.0-70.0, more preferably 55.0-68.—and most preferably 58.6-65.6 degrees Celsius is tested. The resultant difference in Cq value (ΔCq) over the temperature range on the same sample is preferably less than 6, more preferably less than 2, most preferably less than 1. Based on the robustness assay, suitable primers were chosen for the commercial phage test kit. Besides having the desired ACq, suitable robust primers are primers that function in a range of matrices without needing DNA extraction.

Suitable primers (and optionally probes) are included in a suitable qPCR reaction mixture and supplied as pre-made, freeze-dried/lyophilized reaction mixtures in suitable reaction vessels (microcentrifuge tubes or strip of microcentrifuge tubes).

### DETAILED DESCRIPTION

The invention provides a quantitative amplification kit for detection and quantification of phage DNA from a lactic acid bacteria infecting phage in a dairy sample, said kit comprising a first primer pair and wherein said first primer pair has a robustness of a delta Cq lower than 1.0 cycle when tested in a temperature range of 55.0-70.0, preferably 55.0-68.0, more preferably 58.6-65.6 degrees Celsius and wherein said first primer pair is directed to a lactic acid bacteria infecting phage which is a lactococcal phage from the subgroup 936, c2 or P335 or a streptococcal phage from the subgroup pac. The annealing temperature (Ta) of the primers is within the temperature range. io Examples of suitable amplification techniques are: polymerase chain reaction (PCR), reverse transcriptase real-time PCR (RT-real-time qPCR), Isothermal amplification methods (like recombinase polymerase amplification (RPA)<loop mediated isothermal amplification (LAMP) or others), nucleic acid sequence based amplification (NASBA), self-sustained sequence replication (3SR), rolling circle amplification (RCA) or ligase chain reaction.

In one aspect, the invention provides a quantitative polymerase chain reaction (qPCR) kit for detection and quantification of phage DNA from a lactic acid bacteria infecting phage in a dairy sample, said kit comprising a first primer pair and wherein said first primer pair has a robustness of a delta Cq lower than 1.0 cycle when tested in a temperature range of 55.0-70.0, preferably 55.0-68.0, more preferably 58.6-65.6 degrees Celsius and wherein said first primer pair is directed to a lactic acid bacteria infecting phage which is a lactococcal phage from the subgroup 936, c2 or P335 or a streptococcal phage from the subgroup pac.

The kit is used to determine the presence of certain DNA phages in a dairy sample, i.e. to detect phage DNA. Additionally, the kit is used to establish the level of certain DNA phages in a dairy sample, i.e. to quantify phage DNA. The kit can also be used to classify the phages present in dairy sample, and hence the invention also provides a kit for detection, quantification and classification of phage DNA from a lactic acid bacteria infecting phage in a dairy sample.

Phage DNA particles are not the same as plaque forming units (PFU). A PFU is visualized with the overlay assay and is the result of a bacteriophage infecting a host leading to lysis of this host. One PFU is the result of multiple bacteriophages that are released upon lysis of the bacterial host, also referred to as burst size. A test kit of the invention detects and quantifies single bacteriophage particles, one bacteriophage infects one lactic acid bacterium. This implies a 1:1 ratio of bacteriophages with bacterial hosts and thereby a correlation of phage particles with acidification. Like in the overlay assay this number is not affected by the phage, hosts or interaction mechanisms between those two affecting burst sizes. Therefore the kit of the invention shows a better correlation to acidification compared to the overlay assay which is also shown in the examples.

The kit as claimed herein is a kit which allows fast analysis of a dairy sample. The term fast refers to an analysis time of less than 2 hours, preferably the result is obtained within 90 minutes and even more preferably results are available within 60 minutes. The fast analysis is in sharp contrast to the conventional plaque assay which takes at least 48 hours. Additionally, the analysis is performed by the dairy (for example cheese) manufacturer himself and does not need the sending of a sample (or samples) as is the case with the overlap assay.

The to be detected and quantified phage DNA is from phages which are capable of infecting lactic acid bacteria, i.e. from a lactic acid bacteria infecting phage.

The lactic acid bacteria infecting phage is a phage which is capable of infecting a lactic acid bacteria. As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non- sporulating, non-respiring, rod-shaped bacilli or cocci. During the fermentation stage, the consumption of lactose by these bacteria causes the formation of lactic acid, reducing the pH and leading to the formation of a protein coagulum. These bacteria are thus responsible for the acidification of milk and for the texture of the dairy product. As used herein, the term “lactic acid bacteria” or “lactic bacteria” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus salivarius thermophilus, Lactobacillus lactis, Bifidobacterium animalis, Lactococcus lactis, Lactobacillus casei, Lactobacillus plantarum, Lactobacillus helveticus, Lactobacillus acidophilus and Bifidobacterium breve. Preferably, the invention provides a quantitative polymerase chain reaction (qPCR) kit for detection and quantification of phage DNA from a Streptococcus and/or Lactococcus infecting phage in a dairy sample, said kit comprising a first primer pair and wherein said first primer pair has a robustness of a delta Cq lower than 1.0 cycle when tested in a temperature range of 55.0-70.0, preferably 55.0-68.0, more preferably 58.6-65.6 degrees Celsius and wherein said first primer pair is directed to a lactic acid bacteria infecting phage which is a lactococcal phage from the subgroup 936, c2 or P335 or a streptococcal phage from the subgroup pac.

A kit as described herein is used to detect and quantify phages in a dairy sample. More in particular, the dairy sample is obtained from a commercial dairy plant, for example a cheese production plant.

A dairy sample which can be tested with a kit according to the invention is for example a starting material for a dairy manufacturer (such as bulk starter media, a bulk starter culture or milk). Alternatively, a dairy sample is an intermediate from a dairy manufacturing process (such as acidified milk). The to be tested dairy sample can also be a waste stream from a dairy manufacturing process (such as whey). Alternatively, the to be tested sample is a finished product (for example cheese or a fermented dairy product such as yogurt). Other examples of a dairy sample which can be tested with a kit of the invention are rinse water or a swab from anywhere in a dairy production process.

Preferably, the dairy sample which is tested with a kit according to the invention is whey, a bulk starter media, a bulk starter cultures, milk, acidified milk, whey powder, rinse water, a swab from dairy processes, cheese or a fermented dairy product. More preferably, the dairy sample which is tested with a kit according to the invention is whey, a bulk starter media, a bulk starter cultures, milk, acidified milk, whey powder, rinse water or a swab from anywhere in a dairy production process.

As used herein the term “rinse water” refers to the liquid resultant from rinsing a fermentation vat after a fermentation cycle or cleaning cycle and forming the start condition in the vat for a next fermentation. If phages are present in there in high levels it could be quite predictive for fermentation failure.

A kit according to the invention comprises a first primer pair and said first primer pair has a robustness of a delta (Δ) Cq lower than 1.0 cycle when tested in a temperature range (including the annealing temperature (Ta)), of 55.0-70.0, preferably 55.0-68.0, more preferably 58.6-65.6 degrees Celsius. The Cq (quantitation cycle) is the cycle in which a signal (for example fluorescence) can be detected above the threshold level. The Cq-value of an unknown sample can be related to the Cq-value of a known quantity which is thereby used to quantify the amount of phage DNA in the unknown sample. The first primer pair of a kit according to the invention is a robust primer. A robust primer is defined herein as having a delta Cq lower than 1.0 cycle when tested with temperature range (including the annealing temperature (Ta)), ranging from 55.0-70.0, preferably 55.0 - 68.0, more preferably 58.6-65.6 degrees Celsius. I.e. robustness of a primer pair is determined by running/testing primers in a temperature gradient (for example 58.6-65.6 degrees Celsius) resulting in a ΔCq (difference between the highest and lowest Cq value in the temperature range) using the same DNA standard or sample containing the target in all reactions.

Preferably, the first primer pair has a delta Cq lower than 1.0 cycle, more preferably lower than 0.9 or lower than 0.8 or lower than 0.7 cycle, most preferably lower than 0.6 or lower than 0.5 cycle when tested with a temperature range of 55.0-70.0, preferably 55.0-68.0, more preferably 58.6-65.6 degrees Celsius. The lower limit of the delta Cq will be higher than 0.

More preferred, the first primer pair has a delta Cq lower than 1.0 cycle, more preferably lower than 0.9 or lower than 0.8 or lower than 0.7 cycle, most preferably lower than 0.6 or lower than 0.5 cycle when tested with a temperature range from 55.0-70.0 degrees Celsius. The lower limit of the delta Cq is higher than 0.

More preferred, the first primer pair has a delta Cq lower than 1.0 cycle, more preferably lower than 0.9 or lower than 0.8 or lower than 0.7 cycle, most preferably lower than 0.6 or lower than 0.5 cycle when tested with a temperature range from 55.0-68.0 degrees Celsius. The lower limit of the delta Cq is higher than 0.

Most preferred, the first primer pair has a delta Cq lower than 1.0 cycle, more preferably lower than 0.9 or lower than 0.8 or lower than 0.7 cycle, most preferably lower than 0.6 or lower than 0.5 cycle when tested with a temperature range from 58.6-65.6 degrees Celsius. The lower limit of the delta Cq is higher than 0.

Preferably, the first primer pair has a delta Cq of lower than:

1.0 cycle when tested with a temperature range from 55.0-70.0 degrees Celsius.

0.9 cycle when tested with a temperature range from 55.0-70.0 degrees Celsius.

0.8 cycle when tested with a temperature range from 55.0-70.0 degrees Celsius.

0.7 cycle when tested with a temperature range from 55.0-70.0 degrees Celsius.

0.6 cycle when tested with a temperature range from 55.0-70.0 degrees Celsius.

0.5 cycle when tested with a temperature range from 55.0-70.0 degrees Celsius.

1.0 cycle when tested with a temperature range from 55.0-68.0 degrees Celsius.

0.9 cycle when tested with a temperature range from 55.068.0 degrees Celsius.

0.8 cycle when tested with a temperature range from 55.0-68.0 degrees Celsius.

0.7 cycle when tested with a temperature range from 55.0-68.0 degrees Celsius.

0.6 cycle when tested with a temperature range from 55.0-68.0 degrees Celsius.

0.5 cycle when tested with a temperature range from 55.0-68.0 degrees Celsius.

1.0 cycle when tested with a temperature range from 58.6-65.6 degrees Celsius.

0.9 cycle when tested with a temperature range from 58.6-65.6 degrees Celsius.

0.8 cycle when tested with a temperature range from 58.6-65.6 degrees Celsius.

0.7 cycle when tested with a temperature range from 58.6-65.6 degrees Celsius.

0.6 cycle when tested with a temperature range from 58.-65.6 degrees Celsius.

0.5 cycle when tested with a temperature range from 58.6 -65.6 degrees Celsius.

In all cases, the lower limit of the delta Cq is higher than 0.

Additionally (i.e. next to being robust) the first primer pair in a kit of the invention is directed to a lactic acid bacteria infecting phage which is a lactococcal phage from the subgroup 936, c2 or P335 or a streptococcal phage from the subgroup pac. I.e. the first primer pair is directed to a conserved region in a lactococcal phage from the subgroup 936, c2 or P335 or a streptococcal phage from the subgroup pac. Suitable examples are provided herein and will be discussed in more detail later.

The term “conserved” as used herein means highly homologous and relates to a genomic region present in all the phages belonging to a certain phage group in which the DNA sequences of the genomic region are highly similar within a phage subgroup, however, distinct from other phage groups. Thus a conserved region in a lactococcal phage from the subgroup 936 is highly homologous within the 936 subgroup but is not present in any other phage group. The term conserved as used in this context refers to genomic regions in different phages (belonging to the same subgroup) having preferably at least 90% nucleotide identity, more preferably a least 95% identity and most preferably at least 98% identity.

The term lactococcal phage from the subgroup 936 as used herein means a phage belonging to the 936 group. A 936 phage can, for example, be identified using a primer pair comprising primers according to SEQ ID NO. 6 and 7 as described herein (preferably resulting in an amplicon of 243 bp, of which the consensus sequence is given as SEQ ID NO: 1).

The term lactococcal phage from the subgroup c2 as used herein means a phage belonging to the c2 group. A c2 phage can, for example, be identified using a primer pair comprising primers according to SEQ ID NO. 8 and 9 as described herein (preferably resulting in an amplicon of 196 bp, of which the consensus sequence is given as SEQ ID NO: 2).

The term lactococcal phage from the subgroup P335 as used herein means a phage belonging to the P335 group. A P335 phage can, for example, be identified using a primer pair comprising primers according to SEQ ID NO. 10 and 11 as described herein (preferably resulting in an amplicon of 123 bp, of which the consensus sequence is given as SEQ ID NO: 3).

The term streptococcal phage from the subgroup pac as used herein means a phage belonging to the pac group. A pac phage can, for example, be identified using a primer pair comprising primers according to SEQ ID NO. 14 and 15 as described herein (preferably resulting in an amplicon of 278 bp, of which the consensus sequence is given as SEQ ID NO: 5). In one of its aspect, a kit according to the invention is a multiplex qPCR kit, i.e. the kit comprises means for detecting and quantifying at least 2 (preferably at least 3, more preferably at least 4) different phage DNAs wherein at least one of said two different phages is a phage which is detected with a first primer pair is directed to a lactic acid bacteria infecting phage which is a lactococcal phage from the subgroup 936, c2 or P335 or a streptococcal phage from the subgroup pac. The second primer pair may be designed to:
detect a lactic acid bacteria infecting phage which is a lactococcal phage from the subgroup 936, c2 or P335 or a streptococcal phage from the subgroup pac which is not detected with the first primer pair, or
detect another lactic acid bacteria phage, for example a phage belonging to the cos, 5093 or 987 group

The invention thus provides a quantitative polymerase chain reaction (qPCR) kit for detection and quantification of phage DNA from a lactic acid bacteria infecting phage in a dairy sample, said kit comprising a first primer pair and wherein said first primer pair has a robustness of a delta Cq lower than 1.0 cycle when tested in a temperature range from 55.0-70.0, preferably 55.0-68.0, more preferably 58.6-65.6 degrees Celsius and wherein said first primer pair is directed to a lactic acid bacteria infecting phage which is a lactococcal phage from the subgroup 936, c2 or P335 or a streptococcal phage from the subgroup pac, wherein the kit further comprises a second primer pair and wherein said second primer pair is directed to a different phage when compared to the first primer pair. Preferably, said second primer pair also has a robustness of a delta Cq lower than 1.0 cycle when tested in a temperature range from 55.0-70.0, preferably 55.0-68.0, more preferably 58.6-65.6 degrees Celsius.

In yet another aspect, the above described multiplex qPCR kit comprises a third primer pair and wherein said third primer pair is directed to a different phage when compared to the first and second primer pair. Preferably, said third primer pair also has a robustness of a delta Cq lower than 1.0 cycle when tested in a temperature range from 55.0-70.0, preferably 55.0-68.0, more preferably 58.6-65.6 degrees Celsius.

In yet a further aspect, the described multiplex qPCR kit comprises a fourth primer pair and wherein said fourth primer pair is directed to a different phage when compared to the first, second and third primer pair. Preferably, said fourth primer pair also has a robustness of a delta Cq lower than 1.0 cycle when tested in a temperature range from 55.0-70.0, preferably 55.0-68.0, more preferably 58.6-65.6 degrees Celsius.

In one aspect, the first primer pair in a kit according to the invention is directed to a lactococcal phage from the subgroup 936. Preferably, the first primer pair is designed such as to amplify a region from a gene encoding structural protein 1 (GenBank: ASZ71906.1). The invention thus provides a quantitative polymerase chain reaction (qPCR) kit for detection and quantification of phage DNA from a lactic acid bacteria infecting phage in a dairy sample, said kit comprising a first primer pair and wherein said first primer pair has a robustness of a delta Cq lower than 1.0 cycle when tested in a temperature range from 55.0-70.0, preferably 55.0-68.0, more preferably 58.6-65.6 degrees Celsius and wherein said first primer pair is directed to a lactic acid bacteria infecting phage which is a lactococcal phage from the subgroup 936, c2 or P335 or a streptococcal phage from the subgroup pac, wherein said first primer pair anneals to the region comprising a gene encoding structural protein 1 (GenBank: ASZ71906.1; SEQ ID NO:16) in a lactococcal phage from the subgroup 936.

In yet another aspect, the first primer pair in a kit according to the invention is able to detect and quantify DNA from a lactococcal phage from the subgroup 936 or c2. Preferably, the resulting amplicon is an amplicon having SEQ ID NO: 1 (936) or SEQ ID NO: 2 (c2) or an amplicon having at least 80 (preferably at least 90) % identity to SEQ ID NO: 1 or 2.

The invention thus provides a kit as described herein, wherein said first primer pair results in an amplicon having at least 90% identify to SEQ ID NO: 1 (936) or at least 90% identity to SEQ ID NO: 2 (c2). Preferably, said first primer pair results in an amplicon having at least 95% identify to SEQ ID NO: 1 (936) or at least 95% identity to SEQ ID NO: 2 (c2). More preferably, said first primer pair results in an amplicon having at least 98% or 99% identify to SEQ ID NO: 1 (936) or at least 98% or 99% identity to SEQ ID NO: 2 (c2).

Preferably, the first primer pair is selected from:

primer pair SEQ ID NO: 6/SEQ ID NO: 7 (936)
primer pair SEQ ID NO: 8/SEQ ID NO: 9 (c2)
primer pair SEQ ID NO: 10/SEQ ID NO: 11 (P335) and
primer pair SEQ ID NO: 14/SEQ ID NO: 15 (pac).

In case of multiplex qPCR, the first and second primer pair may be selected from:

primer pair SEQ ID NO: 6/SEQ ID NO: 7 (936)
primer pair SEQ ID NO: 8/SEQ ID NO: 9 (c2)
primer pair SEQ ID NO: 10/SEQ ID NO: 11 (P335) and
primer pair SEQ ID NO: 14/SEQ ID NO: 15 (pac).

In case of multiplex PCR, the first, second and third primer pair may be selected from:

primer pair SEQ ID NO: 6/SEQ ID NO: 7 (936)
primer pair SEQ ID NO: 8/SEQ ID NO: 9 (c2)
primer pair SEQ ID NO: 10/SEQ ID NO: 11 (P335) and
primer pair SEQ ID NO: 14/SEQ ID NO: 15 (pac).

In one its aspect, the qPCR kit of the invention is a real-time qPCR kit.

The qPCR kit of the invention does not only comprise a first primer pair but preferably comprises at least one other component as well, selected from:

a DNA polymerase
dNTPs
magnesium, for example magnesium chloride or magnesium sulphate
probe, and
DNA binding dye

Preferably, the DNA polymerase is an inhibitor tolerant DNA polymerase such as SsoAdvanced (Biorad) or PlatinumTag (Thermofisher), PowerUp (Thermofisher) or BiomemeTaq (Promega).

Preferably, the probe anneals to the target DNA and the probe comprises a reporter dye or a reporter dye and a quencher. Preferably, the reporter dye is a fluorophore. Examples of a suitable fluorophore are 6-carboxylfluorescein (FAM), hexachloro-fluorescein (HEX), 6-carboxy-4′5′-dichloro-2′, 7′-dimethoxyfluorescein (JOE), or tetrachlorofluorescein (TET). Example of a suitable quencher is tetramethylrhodamine (TAMRA). A well-known example of a real-time PCR technique which makes use of a probe is TaqMan.

Preferably, the DNA binding dye is a dimeric dye such as, but not limited to, EvaGreen. Alternatively, the DNA binding dye is a monomeric dye such as SYBR-Green or any other asymmetrical cyanic dye.

Additionally, a kit according to the invention comprises an instruction manual. In one of its aspects, the instruction manual comprises instructions to not extract or purify the DNA from the dairy sample. In yet another aspect, the instruction manual comprises instructions to dilute the dairy sample, preferably to dilute the dairy sample with water and even more preferably to dilute the dairy sample with tapwater. Preferably, the dairy sample is diluted at least 10 times, for example by mixing 5 ml of sample with water to a total volume of 50 ml or any other equivalent which results in a dilution of the dairy sample by a factor 10.

Preferably, the different components of the kit are in a lyophilized form allowing storing at ambient temperatures.

The invention further provides a method for detecting and quantifying phage DNA from a lactic acid bacteria infecting phage in a dairy sample, comprising the steps of obtaining a dairy sample
(ii) optionally diluting the obtained dairy sample
(ii) optionally diluting the obtained dairy sample
(iii) testing the, optionally diluted, sample with an qPCR kit as described herein.

The dairy sample can be any of the dairy samples which is described above in the context of the kit. Typically, a dairy sample is taken at a dairy manufacturer such as a cheese or yogurt manufacturer. Preferably, a sample is taken at a cheese manufacturer. Preferably the cheese manufacturer produces cheese on large scale, i.e. a manufacturer which produces at least 3000 kg cheese per year. Or alternatively, a sample is taken from a batch or fermentation vat or fermentation vessel comprising at least 50 L of material. Yet another source of the sample is a sample from a(n) (original) pack size of at least 10 kg of powder, for example whey powder.

Preferably the method for detecting and quantifying phage DNA from a lactic acid bacteria infecting phage in a dairy sample is performed at the dairy manufacturer, i.e. the sample does not to be transported to a test lab outside of the dairy factory.

Preferred samples taken in step (i) are whey, a bulk starter media, a bulk starter cultures, milk, acidified milk, whey powder, rinse water, a swab from dairy processes, cheese or a fermented dairy product.

The obtained sample can be tested as such, for example rinse water can be tested as such and does not need a dilution step. Also after dissolving whey powder in water at an appropriate concentration, the whey powder sample can be tested as such and does not need a dilution step. Also after processing a swab sample in water, the sample can be tested as such and does not need a dilution step.

Other obtained samples need to be diluted, such as whey, milk, acidified milk, a fermented dairy product or bulk starter broth or media.

In optional step (ii) the sample is preferably diluted with water such as tap water, distilled water, double-distilled water or molecular grade water (e.g. MilliQ). Preferably, the dairy sample is diluted with tap water. Buffers which are qPCR compatible can also be used as a means for diluting the dairy sample.

Preferably, the dairy sample is (if at all) diluted at least ten-fold, meaning that x ml sample is diluted such as to end at x0 ml of diluted sample (for example 5 ml sample being diluted with help of 45 ml water to a total volume of 50 ml). Such a dilution can easily be performed by the factory worker by using a scoop to hold 5 mL and put it into a tube which has a visible mark at 50 ml allowing an easy dilution step. Another option would be that the factory worker uses a micropipette, which pipettes a fixed volume, or a pastette to pipette 20 microliters of a dairy sample to a 5 mL tube containing 2.48 mL of water, in this way diluting the sample 125-fold.

Subsequently, 20 to 100 microliters of the diluted sample is transferred to a reaction vessel with freeze-dried “phage test reaction mixture” with a pastette or micropipette. The to be interrogated reaction vessels are transferred to a suitable qPCR cycler with for example a fluorescence reader such as Biorad CFX system or a suitable mobile device such as three9TM (Biomeme).

Preferably, a method according to the invention uses a portable device in step (iii). More preferably the portable device is a portable device with a display on which the phage risk level is io displayed. I.e. the portable device translates the results of the qPCR analysis into an advice for the dairy manufacturer, for example to use another rotation of lactic acid bacteria.

Preferably, a method according to the invention does not comprise a DNA extraction step or a DNA purification step.

A method according to the invention is a fast method meaning that the detection and quantification is finished within a couple of hours. Preferably, step (iii) of said method is completed within 2 hours, preferably within 90 minutes and more preferably within 60 minutes.

Within the context of the present invention, the detection of the PCR product is not based on the size of the amplicon and also not based on gel electrophoresis.

The invention also provides use of a qPCR kit according to the invention for detecting and quantifying phage DNA of lactic acid bacteria infecting phages.

The present invention is further illustrated with non-limiting examples.

### MATERIALS AND METHODS

### 1. Bacterial Strains and Growth Conditions

L. lactis and S. thermophilus (host) strains listed in Table 1 were routinely grown on 10% Reconstituted Skimmed Milk (RSM) overnight at incubation temperature (IT), (32° C. for L. lactis strains and 42° C. for S. thermophilus strains) in PDM broth (yeast extract 10g/l, Bacto peptone 6g/l, Na-b-glycerophosphate 10g/l, lactose 5g/l, 0,6 M MgSO₄ 1,67 ml/l, glycine 2.5g/l) for phage propagation or 10% RSM (Skimmed milk powder 100g/l) for the overlay assay. For strain conservation, one millilitre of overnight culture on 10% RSM was transferred to cryotubes and stored at −80° C.

TABLE 1
Lactic acid bacteria strains used in examples

| Strain | Species | Source |
|---|---|---|
| DS 71733 | L. lactis | DSM, the Netherlands |
| DS 64982 | L. lactis | DSM, the Netherlands |
| DS 73973 | L. lactis | DSM, the Netherlands |
| DS 71732 | L. lactis | DSM, the Netherlands |
| DS 71751 | L. lactis | DSM, the Netherlands |
| DS 71766 | L. lactis | DSM, the Netherlands |
| DS 71600 | S. thermophilus | DSM, the Netherlands |
| DS 64990 | S. thermophilus | DSM, the Netherlands |
| DS 63626 | L. lactis | DSM, the Netherlands |
| DS 74031 | L. lactis | DSM, the Netherlands |

### 2. Bacteriophage Assays

Spot assays were performed by seeding the PDM semi-solid agar overlay with 300 μl fresh Overnight Culture (ON) and applying 10 μl of phage lysate in a grid format, as described by Dupont, K., et al, J. (2005, J Appl Microbiol, 98, 1001-1009. Plates were then allowed to dry and incubated anaerobically ON at IT. A clear zone was assumed to indicate phage mediated lysis of the bacterial lawn by the applied phage and was recorded as ‘+’, whereas absence of lysis was recorded as

### 3. Phage Propagation and Enumeration

Whey samples from dairy plants producing fermented milk products were obtained and analysed for the presence of phages against lactic acid bacteria strains listed as host in Table 1 using the spot assay described above under “Bacteriophage assays”. Defined single plaques were isolated by twice single plaque purification on semi-solid overlays. Phages were then propagated as follows: 25 ml PDM broth (yeast extract 10 g/I, Bacto peptone 6 g/l, Na-b-glycerophosphate 10 g/l, lactose 5 g/l, 0,6 M MgSO₄ 1,67 ml/l, glycine 2.5 g/l) was inoculated (1%) with a fresh ON culture of the appropriate host strain and incubated at IT for 1.0-2.5 hours. Then, a single plaque was added to the growing culture, mixed well and incubated for a further 2-4 hours. The lysed culture was centrifuged and the supernatant filtered (0.45 pm). The filtered supernatant was used as the phage stock for subsequent assays. Table 2 summarizes the phages that were obtained in this manner.

TABLE 2
bacteriophages used in examples

| Phage | Species | Description | Source |
|---|---|---|---|
| 71733-D3 | P335 | Virulent phage of | DSM, The Netherlands |
| | | L. lactis DS 71733 | |
| 64982-D2 | P335 | Virulent phage of | DSM, The Netherlands |
| | | L. lactis DS 64982 | |
| 73973-D1 | 936 | Virulent phage of | DSM, The Netherlands |
| | | L. lactis DS 73973 | |
| 71732-D1 | 936 | Virulent phage of | DSM, The Netherlands |
| | | L. lactis DS 71732 | |
| 71751-D6 | c2 | Virulent phage of | DSM, The Netherlands |
| | | L. lactis DS 71751 | |
| 71766-D2 | c2 | Virulent phage of | DSM, The Netherlands |
| | | L. lactis DS 71766 | |
| 71600-D1 | Cos | Virulent phage of | DSM, The Netherlands |
| | | S. thermophilus DS 71600 | |
| 64990-D1 | Pac | Virulent phage of | DSM, The Netherlands |
| | | S. thermophilus DS 64990 | |
| 63626-D1 | 936 | Virulent phage of | DSM, The Netherlands |
| | | L. lactis DS 63626 | |
| 63626-D3 | C2 | Virulent phage of | DSM, The Netherlands |
| | | L. lactis DS 63626 | |
| 74031-D2 | C2 | Virulent phage of | DSM, The Netherlands |
| | | L. lactis DS 74031 | |

### 4. Bacteriophage DNA Isolation and Sequencing

Individual phages as listed in Table 2 were propagated in a 2-liter volume before concentration by polyethylene glycol (PEG) 8000 (Sigma-Aldrich) precipitation and purification using a discontinuous cesium chloride (CsCI; Sigma-Aldrich) block gradient as described by Sambrook et al. (23), using a Beckman 50 Ti rotor (Beckman Coulter, Brea, Calif., USA). Phage DNA was prepared using a method adapted from Moineau et al. (22) and Sambrook et al. (23). Briefly, 20 μl proteinase K (20 mg/ml; Fisher Scientific, Waltham, Mass., USA) was added to 500 μl of CsCI purified phage, and the mixture was heated at 56° C. for 20 min. A sodium dodecyl sulfate solution (SDS; Sigma-Aldrich) o was then added to a final concentration of 1.5% before heating at 65° C. for 30 min. Potassium acetate was added to a final concentration of 1 M, and the mixture was placed on ice for 30 min. Centrifugation at 13,200×g for 10 min was followed by two phenol-chloroform-isoamyl alcohol (25:24:1; Sigma-Aldrich) extractions and the addition of 0.1 volume of 3M sodium acetate (pH 4.8; Lancaster Synthesis, Ward Hill, Mass., USA) and 2.5 volumes of ice-cold 96% ethanol. Precipitated phage DNA was pelleted at 21,000×g for 15 min and resuspended in 50 μl Tris-EDTA (TE) buffer (10 mM Tris-HCl, 1 mM EDTA [Sigma-Aldrich]; pH 7.5). Phage DNA was visualized on 1% agarose (Sigma-Aldrich) gels stained with Midori Green Advance DNA stain (Nippon Genetics Europe GmbH, Dueren, Germany) using the method of Sambrook et al. (1989, Molecular cloning: a laboratory manual, 2nd ed.). Approximately 20 pg phage DNA was extracted and verified by nanodrop (Nanodrop 2000; Thermo Scientific) quantification. Confirmatory molecular identification (ID) tests were also conducted on the DNA extract prior to shipment to the contract sequencing facility (Macrogen Inc., Seoul, South Korea). At least 100-fold sequencing coverage was obtained using pyrosequencing technology on a 454 FLX instrument. The individual sequence files generated by the 454 FLX instrument were assembled with GSassembler (454 Lifesciences, Branford, Conn., USA) to generate a consensus sequence. Quality improvement of the genome sequence involved Sanger sequencing (Eurofins MWG, Ebersberg, Germany) of at least three PCR products across each entire genome to ensure correct assembly, double stranding, and the resolution of any remaining base conflicts occurring within homopolymertracts. Genomes were annotated using a heuristic approach (Genemark) Besemer J, et al. (1999, Nucleic Acids Res 27:3911-3920) and manually using the Basic Local Alignment Search Tool (NCBl). Conserved protein domains (where relevant) were detected using Pfam, Sonnhammer EL et al. (1997, Proteins 28:405-420), HHpred, Soding J, et al. (2005, Nucleic Acids Res 33:W244-W248) and/or CDD, Marchler-Bauer et al. (2015, Nucleic Acids Res 43:D222-D226. Complete genomes were visualized using Artemis, Rutherford K, et al. (2000, Bioinformatics 16:944-945.).

### 5. In Silico Primer Design

Oligonucleotide primers were developed according a specific workflow, this included, in silico primer development and in vitro protocol optimization. For in silico primer development sequences obtained from complete bacteriophage genomes from public- and in-house databases, were used to perform multiple sequence alignments using Geneious (Biomatters Ltd., New Zealand, version 10.1.3) to locate conserved regions for primers and or probes. Several primer sets were designed o using the online primer3 tool with specifically in mind an annealing temperature of 60° C. and a product of approximately 200 base-pairs. With a multi primer analyzer tool (ThermoScientific, USA) using the most stringent criteria, primers with lowest chance to form primer dimers or self-dimers were selected. The primers' specificity was tested in silico using primer-BLAST (NCBl, USA). Oligonucleotides were synthesized at Integrated DNA Technologies (IDT, Germany). Thereafter in vitro tests (qPCR) were performed to select the most robust primer set. First a gradient test was performed in a temperature range from 58.6 to 65.6 ° C. The primer set with the lowest ΔCq-range (below 1.0, preferably lower), was selected and subjected to specificity tests. After primer specificity was confirmed the primer concentrations were optimized. Thereafter, method development was finalized by running standard curves. qPCR tests were performed as described below in Real-time quantitative PCR assays.

### 6. Bacteriophage DNA Isolation for qPCR Analysis

Lysates from phages listed in Table 2 were used for genomic DNA extraction for sequencing and qPCR. Genomic DNA extraction was performed with a Phage DNA isolation kit (Norgen Biotek Corp., Canada). DNA quantity was measured using a Qubit Fluorometer and dsDNA BR Assay kit,

DNA quality was assessed by Nanodrop measurement. DNA concentration combined with genome information was then used to calculate the number of isolated phage DNA particles using the following formula:

Phage particles=DNA concentration (g)×((Avogadro constant+(genome (bp)×650). After dilution in ddH₂0 genomic DNA was used as template in qPCR assays.

### 7. Real-Time Quantitative PCR Assay

Real-time PCR assays were performed on a CFX96 system (Biorad, USA). Reactions were run using the following thermocycling conditions: hot start at 95° C. for 3 min; followed by 40 cycles of (i) 95° C. for 10 sec, (ii) 60° C. for 20 sec., ensuring phage detection within 50 minutes, subsequent to the amplification, a melting curve analysis was performed. qPCR reactions were performed in a total volume of 25 μl compromising of a volume of a 2× qPCR mastermix (SsoAdvanced™ Universal Inhibitor-tolerant SYBRgreen® Supermix, Hercules, USA), a forward and reverse primer (concentration 50-400 nM), ddH20 and phage template. Phage DNA template was either: phage DNA, phage lysate or phage in a dairy matrix. qPCR reactions were performed in appropriate 96 well plates (Biorad, Hercules, USA). In order to determine the number of phage particles in a reaction template Cq values were obtained by setting a threshold in the exponential phase of a reaction. This threshold preferably was fixed depending on the assay used and standard curve that was generated using a 10-fold dilution series of phage particles (genomic DNA sample) analyzed in five-fold. The Cq-value of an unknown samples was then calculated back to a number of phage particles using the pre-determined standard curve.

### 8. Acidification Experiments

Acidification experiments were performed as follows. (i) Day 1: 15 ml tubes with 12 ml RMS 10% was inoculated with 2% w/v from a cryotube. Culture was incubated overnight. (ii) Day 2: preparation of CINAC starter: at the end of the day 50 ml greiner tubes with 25 ml RSM 10% was inoculated with 2% w/v 0/N culture. Starter was incubated overnight. (iii) Day 3: greiner tubes with 35 ml RSM 10% were inoculated with 1% w/v starter. For experiments with LL-50 rotations the inoculation was based on unit weight. This was called T0. Prior to inoculation the starter was diluted two times. At T0, the cultures were infected with bacteriophages. After mixing by decanting a 50 ml greiner tube the lid was removed and tubes were covered with parafilm. The tube was placed in a pre-warmed water bath after which a pH-probe was added. Acidification experiments were performed in a water bath set at 32° C. and the pH was monitored for 24 hours.

### EXAMPLE 1

### In silico Design of Primers to Conserved Regions of Phage Genome Sequences

Since milk and other relevant dairy samples are known to contain inhibitory compounds to the qPCR assay (e.g. annealing of primer and DNA polymerase complex, elongation of DNA strand by DNA polymerase) the so-called robustness of developed primer sets is of utmost relevance to ensure efficient amplification, and, thereby, proper quantification of the amplicon. Robustness of primer sets is determined by determining the difference in Cq value after quantification of the same sample in a temperature gradient.

936

Using 17 bacteriophage genomes for multiple alignment selected from the public domain (NCBl) and DSM collection, conserved regions were found. In the 936-genome alignment a novel conserved gene, i.e. a gene encoding structural protein 1 (for example, coding for protein GenBank: ASZ71906.1; SEQ ID NO: 16), was identified as a suitable target for primer/probe development. The amplicon generated by the primer set SEQ ID NO: 6 and SEQ ID NO: 7 is sized 243 bp (SEQ ID NO: 1) covering a 264 bp gene annotated as hypothetical structural protein and some addition base pairs.

TABLE 3
936 BLAST results identity

| | | | Am- | F Primers | R Primers | Amplicon |
|---|---|---|---|---|---|---|
| | 936-F1 | 936-R1 | plicon | prior art | prior art | prior art |
| | This in- | This in- | This in- | Labrie⁽¹⁾ | Labrie⁽¹⁾ | Labrie⁽¹⁾ |
| | vention | vention | vention | Verrault⁽²⁾ | Verrault⁽²⁾ | Verrault⁽²⁾ |
| Identity | 100-96 | 100-95 | 100-97 | 100-95⁽¹⁾ | 100-95⁽¹⁾ | 98-93⁽¹⁾ |
| (%) | | | | 100-95⁽²⁾ | 100-95⁽²⁾ | 100-83⁽²⁾ |

⁽¹⁾Labrie and Moineau (2000, Appl Environ Microbiol, 66, pp. 987-994)
⁽²⁾Verreault et al. (2011 Detection of airborne lactococcal bacteriophages in cheese manufacturing plants. Appl Environ Microbiol. 77: 491-497)

It was confirmed by pairwise comparison that there was a minimum of 95% identity between primers and virtually all analogues sequences on the target genes accessible at the time of filing of this application. Homology in the target regions showed a similar number, with in between 97% and 100% identity.

c2

Using 14 bacteriophage genomes for multiple alignment selected from the public domain (NCBI) and DSM collection, conserved regions were found. In the c2 genome alignment a new region was identified in a gene also used in publications. This new region was a region of 360 bp of a total number of 1492 bp in a gene annotated as the major capsid protein. On the new region primer (SEQ ID NO: 8 and SEQ ID NO: 9 were designed having an amplicon size of 196 bp (SEQ ID NO 2).

TABLE 4
c2 BLAST results identity

| | | | | F Primers | R Primers | Amplicon |
|---|---|---|---|---|---|---|
| | c2-F3 | c2-R3 | Amplicon | prior art | prior art | prior art |
| | This invention | This invention | This invention | Labrie⁽¹⁾ | Labrie⁽¹⁾ | Labrie⁽¹⁾ |
| | | | | Verrault⁽²⁾ | Verrault⁽²⁾ | Verrault⁽²⁾ |
| Identity | 100 | 100 | 100-95 | 100-97⁽¹⁾ | 100-93^{*2 (1)} | 100-91⁽¹⁾ |
| (%) | | | | 100-95^{*1(2)} | 100⁽²⁾ | 100-90⁽²⁾ |

^{*1}detects only 10 of 14 genomes used in alignment.
^{*2}detects only 11 of 14 genomes used in alignment.
⁽¹⁾Labrie and Moineau (2000, Appl Environ Microbiol, 66, pp. 987-994)
⁽²⁾Verreault et al. (2011 Detection of airborne lactococcal bacteriophages in cheese manufacturing plants. Appl Environ Microbiol. 77: 491-497)

It was confirmed by pairwise comparison that there was a minimum of 95% identity between primers and virtually all analogues sequences on the target genes accessible at the moment. Homology in the target region showed a similar number, and in between 93% and 100% identity.

P335

Using 17 bacteriophage genomes for multiple alignment selected from the public domain (NCBI) and DSM collection, the most conserved gene in p335 phages, encoding a dUTPase, was used for robust primer development. On the dUTPase gene covering 420 bp primers (SEQ ID NO: 10 and SEQ ID NO: 11) were designed having an amplicon size of 123 bp (SEQ ID NO 3).

TABLE 5
p335 BLAST results identity

| | | | | F Primers | R Primers | Amplicon |
|---|---|---|---|---|---|---|
| | p335-F4 | P335-R4 | Amplicon | prior art | prior art | prior art |
| | This | This | This | Muhammed⁽³⁾ | Muhammed⁽³⁾ | Muhammed⁽³⁾ |
| | invention | invention | invention | | | |
| Identity (%) | 100 | 100-95 | 100-94 | 100-94⁽³⁾ | 100⁽³⁾ | 100-96⁽³⁾ |

⁽³⁾Muhammed et al. (2017, PLoS One., A high-throughput qPCR system for simultaneous quantitative detection of dairy Lactococcus lactis and Leuconostoc bacteriophages, 12: e0174223)

Pac

Using 14 bacteriophage genomes for multiple alignment selected from the public domain (NCBl) and DSM collection, a conserved region was identified that was not used before for developing robust primers. This new region consisted of 314 bp covering a part of a 596 bp gene annotated as putative scaffold protein. In the new region primers (SEQ ID NO: 14 and SEQ ID NO: 15) were developed having an amplicon size of 278 bp (SEQ ID NO: 5).

TABLE 6
Pac BLAST results identity

| | | | Am- | F Primers | R Primers | Amplicon |
|---|---|---|---|---|---|---|
| | pac-F2 | pac-R2 | plicon | prior art | prior art | prior art |
| | This in- | This in- | This in- | Del Rio⁽⁴⁾ | Del Rio⁽⁴⁾ | Del Rio⁽⁴⁾ |
| | vention | vention | vention | | | |
| Identity | 100 | 100 | 100-85 | 100-90⁽⁴⁾ | 100-92⁽⁴⁾ | 100-82⁽⁴⁾ |
| (%) | | | | | | |

⁽⁴⁾Del Rio et al. (2008, Appl Environ Microbiol., Multiplex fast real-time PCR for quantitative detection and identification of cos- and pac-type Streptococcus thermophilus bacteriophages, 74: 4779-4781)

Conserved regions in phages were very limited. It is reasonable to assume that the higher the identity in a region the chance increases for primers to target more and thus yet to be identified phages genomes belonging to the same group and over a longer period guarantees performance of an assay, as the chance is low mutations while arise in such a region that could lead to failure of the assay.

### EXAMPLE 2

### Newly Developed Primer Sets for 936, c2, P335 and Pac Tested on Phage DNA have increased Robustness over Prior Art

Robustness of a primer set was determined by running primers in a temperature gradient resulting in a ΔCq (difference between the highest and lowest Cq value in the range). A robust primer had a ΔCq preferably as low as possible, however the maximum value that was accepted was below or equal to 1.0. The ΔCq of robust primers developed by the inventors were compared with the ΔCq of prior art primers in a temperature gradient ranging from 58.6° C. to 65.6° C. For a robust qPCR in milk besides robust primers also a robust polymerase was required. The selected polymerase was the SsoAdvanced™ Universal Inhibitor-tolerant SYBRgreen® Supermix.

TABLE 7
Robustness

| | 936 | 936 | c2 | c2 | P335 | P335 | | Pac |
|---|---|---|---|---|---|---|---|---|
| Primers | A/B | F1/R1 | A/B | F3/R3 | A/B | F4/R4 | Pac | F2/R2 |
| Reference | Labrie | This | Labrie | This | Labrie | This | Del Rio | This |
| | and | invention | and | invention | and | invention | (2008) | invention |
| | Moineau | | Moineau | | Moineau | | | |
| | (2000) | | (2000) | | (2000) | | | |
| SEQ ID NO | | 6/7 | | 8/9 | | 10/11 | | 14/15 |
| ΔCq Range | 10.75 | 0.49 | 6.79 | 0.32 | 7.44 | 0.70 | 9.21 | 0.52^{(*)} |

^{(*)}Temperature range 58.6-64.5

DSM primers were robust, in contradiction to prior-art primers which were not. This is shown with the ΔCq range of all DSM primers being below 1.0. The robust primer requirement for specifically related to be able to analyze samples in a complex (dairy) matrix as which is shown in example 4. The primers also complied to other criteria as described previously and is shown in the example 3.

### EXAMPLE 3

### qPCR Assay Performance 936, c2 and Calibration

qPCR assay performance was determined by running standard curves using bacteriophage DNA as described in material and methods. Additionally, the qPCR assays were calibrated by analysing a tenfold serial dilution of phage lysate with the qPCR and overlay assay.

TABLE 8
Results Standard curves 936 and c2.

| | | | | | | | |
|---|---|---|---|---|---|---|---|
| | | LOG | | | | | |
| Dilution | Particles/ml | particles/ml | Cq 1 | Cq 2 | Cq 3 | Cq 4 | Cq5 |

936

| | | | | | | | |
|---|---|---|---|---|---|---|---|
| 1.00E+00 | 1.63E+11 | 11 | 7.26 | 7.14 | 7.00 | 7.21 | 7.15 |
| 1.00E−01 | 1.63E+10 | 10 | 10.58 | 10.52 | 10.52 | 10.63 | 10.47 |
| 1.00E−02 | 1.63E+09 | 9 | 14.13 | 14.19 | 14.16 | 14.18 | 14.11 |
| 1.00E−03 | 1.63E+08 | 8 | 17.51 | 17.58 | 17.53 | 17.54 | 17.51 |
| 1.00E−04 | 1.63E+07 | 7 | 21.22 | 21.07 | 21.00 | 21.08 | 20.92 |
| 1.00E−05 | 1.63E+06 | 6 | 24.51 | 24.30 | 24.39 | 24.44 | 24.39 |
| 1.00E−06 | 1.63E+05 | 5 | 27.96 | 28.05 | 28.19 | 28.12 | 28.15 |
| 1.00E−07 | 1.63E+04 | 4 | 31.27 | 31.20 | 31.08 | 31.39 | 31.8 |
| 1.00E−08 | 1.63E+03 | 3 | 36.36 | 35.00 | 35.14 | 35.15 | 34.47 |
| NTC | | | N/A | N/A | N/A | N/A | N/A |

c2

| | | | | | | | |
|---|---|---|---|---|---|---|---|
| 1.00E+00 | 4.83E+11 | 12 | 5.81 | 5.5 | 5.45 | 5.56 | 5.47 |
| 1.00E−01 | 4.83E+10 | 11 | 9.16 | 9.18 | 9.05 | 9.11 | 9.05 |
| 1.00E−02 | 4.83E+09 | 10 | 12.73 | 12.78 | 12.82 | 12.86 | 12.67 |
| 1.00E−03 | 4.83E+08 | 9 | 16.16 | 16.09 | 16.15 | 16.16 | 16.05 |
| 1.00E−04 | 4.83E+07 | 8 | 19.56 | 19.57 | 19.45 | 19.48 | 19.5 |
| 1.00E−05 | 4.83E+06 | 7 | 22.97 | 23.02 | 22.88 | 22.78 | 22.91 |
| 1.00E−06 | 4.83E+05 | 6 | 26.24 | 26.46 | 26.47 | 26.20 | 26.32 |
| 1.00E−07 | 4.83E+04 | 5 | 29.81 | 29.52 | 29.82 | 30.01 | 29.56 |
| 1.00E−08 | 4.83E+03 | 4 | 32.77 | 32.98 | 34.01 | 33.09 | 33.06 |
| NTC | | | N/A | N/A | N/A | N/A | N/A |

The standard curve was used to determine qPCR performance parameters of which the efficiency was calculated using the following formula:

$$Efficiency = ( {10}^{( \frac{- 1}{Slope} ) - 1} ) * 100$$

TABLE 9
qPCR performance

| Parameter | 936 F1/R1 | c2 F3/R3 |
|---|---|---|
| Slope | −3.4909 | −3.4387 |
| Intercept | 46.23 | 45.89 |
| R2 | 0.9998 | 0.9999 |
| Efficiency | 93.4% | 95.3% |
| Linear dynamic | 9Log10 | 9Log10 |
| range | | |
| NTC | — | — |

The 936 and c2 qPCR performance parameters show a broad linear dynamic range and an efficiency between 90 and 100%.

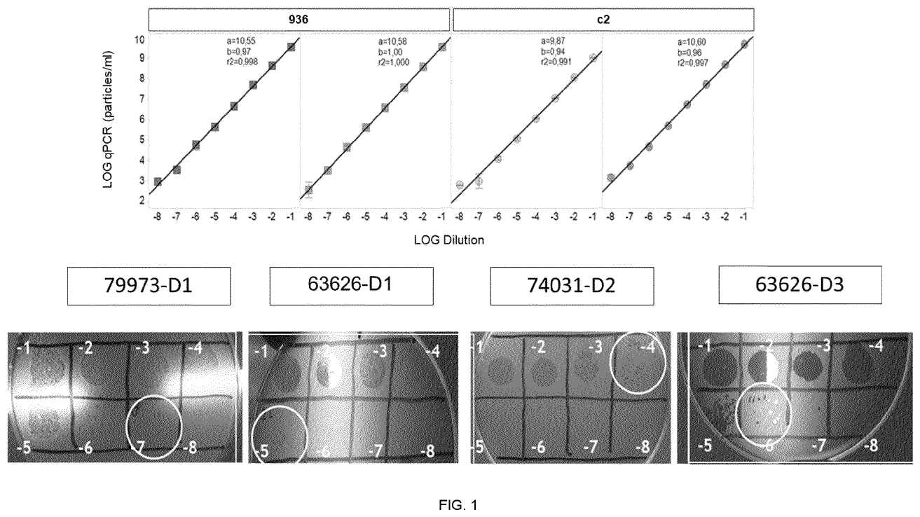

Both 936 and c2 qPCR assays were calibrated using two 936 and two c2 phage lysates. From the lysates ten-fold serial dilutions were prepared in saline and analyzed using the overlay assay and qPCR, results are shown in FIG. 1. The results in FIG. 1 clearly show the qPCR detected phage particles even in the lowest dilutions, while the overlay could not. The difference between detected dilutions ranged from a factor 10, to a factor 10.000. Furthermore, the qPCR results were consistent for different phages belonging to the same species and even between different species.

### EXAMPLE 4

### Measurement of Phage Titers During Acidification with 936 and c2 qPCR Assays and Overlay

Previous examples showed that the developed qPCR assays showed the required sensitivity and a higher robustness (when comparing the ACq in a robustness test) than assays developed in the prior-art. To show the performance of the developed robust qPCR assays during acidification of milk, the example below describes the measurement of phage titers by qPCR and overlay assay on samples taken from lab scale fermentations of L. lactis spiked with a known amount of virulent phages. Acidification experiments were performed as described above in Material and Methods and Table 10 below shows the strains used, the bacteriophage used for infection and its spiked titer (PFU/mL as determined by overlay assay).

TABLE 10
overview of the different acidification experiments on 10% RSM with L. lactis host strain
DS 63626 with or without phage infected at T0 of the fermentation. Asterisk (*) indicates the
particular phage has an established virulence against the host strain used in acidification
experiment. Performance was scored as: OK, the time to reach (TTR) pH 5.2 was between 6 and
6.5 hours similar as the control fermentations 11 and 12; slow down, TTR pH 5.2 was greater than
6.5 hours; Failure, pH 5.2 was not reached within 24 hours (number between brackets indicates
time in hours when failure started to occur).

| | | | Spiked titer at T0 | |
|---|---|---|---|---|
| Exp.# | phage | species | (PFU/mL) | Performance |
| 1 | 63626-D1* | 936 | 10 | Failure (4 h) |
| 2 | 63626-D1* | 936 | 10 | Failure (4 h) |
| 3 | 63626-D1* | 936 | 1000 | Failure (3 h) |
| 4 | 63626-D1* | 936 | 1000 | Failure (3 h) |
| 5 | 63626-D3* | C2 | 10 | Slowdown, pH 5.2 > hours |
| 6 | 63626-D3* | C2 | 10 | Failure (4 h) |
| 7 | 63626-D3* | C2 | 1000 | Failure (3 h) |
| 8 | 63626-D3* | C2 | 1000 | Failure (3 h) |
| 9 | 63626-D1* | 936 | 1000 | Failure (3 h) |
| | 74031-D2 | C2 | 1000 | |
| 10 | 74031-D2 | C2 | 1000 | OK |
| 11 | No phage | — | — | OK (control) |
| 12 | No phage | — | — | OK (control) |

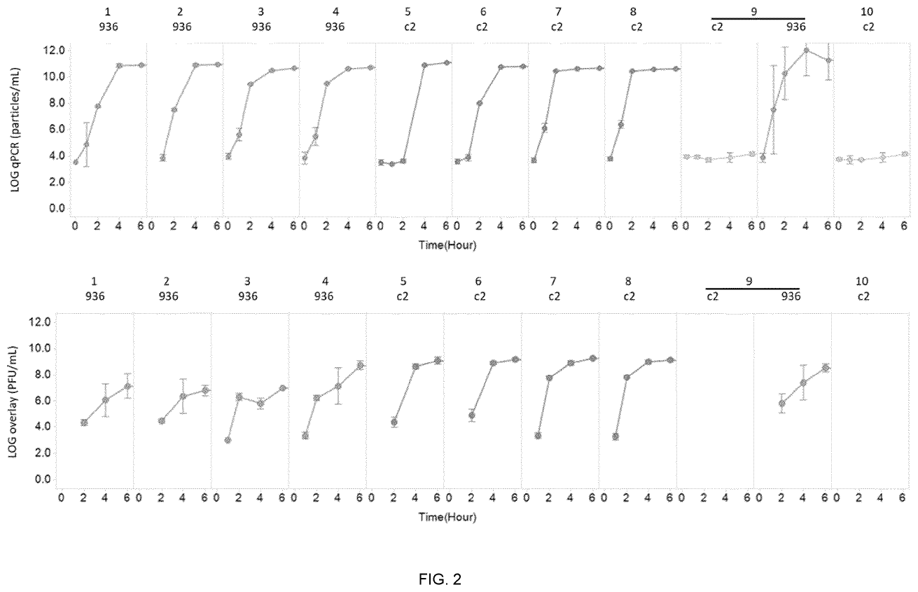

At T0 and after 1, 2, 4 and 6 hours samples were taken from the acidifications and diluted 10-fold in MilliQ to measure with qPCR or with overlay assay. For qPCR assays with either primers for 936 (SEQ ID NO: 6 and SEQ ID NO: 7), or c2 (SEQ ID NO:8 and SEQ ID NO: 9) were used depending which phage species were spiked in the acidification experiment. The phage titer as determined by qPCR was determined by extrapolation of the Cq value on the standard curve and expressed as particles/mL. For the overlay assay, further dilutions were spotted on a top agar bacterial lawn grown with DS 63626 to determine the phage titer (PFU/mL) of the sample. The phage titers found in the samples with both methods are shown in FIG. 2.

In case of fermentation failures due to the spiking of virulent phages, the rate of phage particle increase as determined by either the 936 or the c2 qPCR assay, corresponded well to the timepoint the fermentation failure became evident, i.e. in case of experiments #1, #2, #3, #4, #6, #7, #8, #9). In those experiments, particles/mL reached a level of more than 10⁸ after 2 hours of fermentation. In the case of experiment #9, where 936 qPCR assay indicated those high levels, the non virulent c2 phase which was also added to that experiment, remained stably low during the acidification (between 10³ and 10⁴ particles/mL), indicating the specificity of the c2 qPCR assay. Interestingly, whereas a fermentation failure was found for experiment #6, a slow-down was observed in duplicate experiment #5. The particle levels in experiment #5 only rose to the levels of 10⁸ particles/mL after 4 hours, whereas this level was almost reached in experiment #6 2 hours earlier. This result indicates a correlation to the extent of acidification issues and the discriminatory level of the qPCR assay. In general, the overlay assay started to indicate phage titers at later timepoints than acidification issues rose or that the qPCR indicated rising phage levels (in most cases at least 2 hours later). Also, when looking at the different outcome of the acidification of experiment #5 vs #6, the overlay assay indicated a similar profile of phage titers whereas the qPCR o assay showed differences hinting at the differential outcome.

These results show that the robust 936 and c2 qPCR assays quantified phage particles in acidified milk samples. Furthermore, the qPCR assays showed a higher sensitivity and a better correlation to the dynamics of phage infection and titer build-up during acidification than the traditional overlay assay.

1. A quantitative polymerase chain reaction (qPCR) kit for detection and quantification of phage DNA from a lactic acid bacteria infecting phage in a dairy sample, said kit comprising a first primer pair and wherein said first primer pair has a robustness of a delta Cq lower than 1.0 cycle when tested in a temperature range of 55.0-70.0, optionally 55.0 68.0, optionally 58.6-65.6 degrees Celsius and wherein said first primer pair is directed to a lactic acid bacteria infecting phage which is a lactococcal phage from the subgroup 9362, c2 or P335 or a streptococcal phage from the subgroup pac.

2. The qPCR kit according to claim 1, wherein the kit further comprises a second primer pair and wherein said second primer pair has a robustness of a delta Cq lower than 1.0 cycle when tested in a temperature range of 55.0-70.0, optionally 55.0 68.0, optionally 58.6-65.6 degrees Celsius and wherein said second primer pair is directed to a different phage when compared to said first primer pair.

3. The qPCR kit according to claim 1, wherein said first primer pair anneals to a region of a gene encoding structural protein 1 (GenBank: ASZ71906.1; SEQ ID NO:16) in a lactococcal phage from the subgroup 936.

4. The qPCR kit according to claim 1, wherein said first primer pair results in an amplicon having at least 90% identify to SEQ ID NO: 1 (936) or at least 90% identity to SEQ ID NO: 2 (c2).

5. The qPCR kit according to any of claim 1, wherein said first primer pair is selected from:
primer pair SEQ ID NO: 6/SEQ ID NO: 7 (936)
primer pair SEQ ID NO: 8/SEQ ID NO: 9 (c2)
primer pair SEQ ID NO: 10/SEQ ID NO: 11 (P335) and
primer pair SEQ ID NO: 14/SEQ ID NO: 15 (pac).

6. The qPCR kit according to claim 1 wherein said qPCR is a real-time qPCR kit.

7. The qPCR kit according to claim 1, further comprising instructions to not extract or purify the DNA from the sample.

8. The method for detecting and quantifying phage DNA from a lactic acid bacteria infecting phage in a dairy sample, comprising
(i) obtaining a dairy sample
(ii) optionally diluting the obtained dairy sample
(iii) testing the, optionally diluted, sample with an qPCR kit according to claims 1.

9. The method according to claim 8 wherein the dairy sample from (i) is obtained from a dairy production batch of at least 50 liters or from a pack size of at least 10 kg of powder.

10. The method according to claim 8, wherein the dairy sample is whey, a bulk starter media, one or more bulk starter cultures, milk, acidified milk, whey powder, rinse water, a swab from dairy processes, cheese or a fermented dairy product.

11. The method according to claim 8, further comprising using a portable device for performing the qPCR analysis.

12. The method according to any onc of claims claims 8, wherein diluting the obtained dairy sample is diluting the obtained dairy sample with water.

13. The method according to any onc of claim 8, which does not comprise DNA extraction or DNA purification.

14. The method according to claim 8, wherein (iii) of said method is completed within 2 hours, optionally within 90 minutes and optionally within 60 minutes, after obtaining the dairy sample.

15. A product comprising a qPCR kit according to claim 1 for detecting and quantifying phage DNA.
